(19) United States
(12) Patent Application Publication
(10) Pub. No.: US 2026/0262563 A1
(43) Pub. Date: Sep. 10, 2026

(54) COMPUTER-IMPLEMENTED METHOD FOR SEEDING, PLANTING AND/OR FERTILIZING

(71) Applicant: BASF AGRO TRADEMARKS GMBH, Ludwigshafen (DE)

(72) Inventors: Christian VON HEBEL, Koeln (DE); Mollie Jo HOSS-KUHNE, Windeck (DE); Bjoern KIEPE, Köln (DE)

(21) Appl. No.: 18/847,074

(22) PCT Filed: Mar. 16, 2023
(86) PCT No.: PCT/EP2023/056803

§ 371 Date: Sep. 13, 2024

(30) Foreign Application Priority Data
Mar. 16, 2022 (EP) 22162544.5

(51) Int. Cl. A01C 21/00; A01C 7/20; G01N 33/24

(52) U.S. Cl. CPC A01C 21/00; A01C 7/203; G01N 33/245

(57) ABSTRACT

A computer-implemented method for seeding, planting and/or fertilizing, comprising: providing soil data by transmitting via transmitting means at least one transmitting signal into at least a part of a soil and by receiving via receiving means a response signal from the soil; providing configuration parameters of the transmitting means and receiving means; determining a soil structure of the soil by a subsurface model based on the soil data and the configuration parameters; determining a seeding, planting and/or fertilizing depth in the determined soil structure; and providing at least one seed, plant and/or fertilizer in the determined seeding, planting and/or fertilizing depth in the soil.

### TECHNICAL FIELD

The present disclosure relates to a computer-implemented method for seeding, planting and/or fertilizing, a device for seeding, planting and/or fertilizing, a system for seeding, planting and/or fertilizing, and use of such a method in an agricultural device, a computer program element, a subsurface model for determining a soil structure, and training data for training the subsurface model.

### TECHNICAL BACKGROUND

The general background of this disclosure is the seeding, planting and/or fertilizing of seeds, plants, and fertilizers in an agricultural field, which may be an agricultural field, a greenhouse, or the like.

It is known in the art, that each plant variety needs specific conditions, e.g. specific amount of water, specific value of solar radiation and/or specific amount of nutrients, in order to grow optimally and to provide the highest amount of crop yield. Conditions which differ from these specific conditions may lead to damages and/or decreased growth of the plants and therefore decreased crop yield. In the conventional farming seeds, plants and/or fertilizers are provided in crop planting rows into a soil volume/area of an agricultural field in an even/homogenous depth. However, the conditions, in particular the water contents and the nutrient contents in the soil, vary greatly both horizontally and vertically in the soil of an agricultural filed. Therefore, conventional farming disadvantageously seeds, plants and/or fertilizes seeds, plants into the soil of an agricultural field, limits the growth of plants which in turn can limit crop yields.

It has been found that a need exists to provide a more precise seeding, planting and/or fertilizing of seeds, plants, and fertilizers in an agricultural field in order to increase the plant growth and fertilizer use efficiency and therefore increase the harvest/crop yield.

### SUMMARY OF THE INVENTION

In one aspect of the present disclosure a computer-implemented method for seeding, planting and/or fertilizing is presented, comprising the steps of
providing soil data by transmitting via transmitting means at least one transmitting signal into at least a part of a soil and by receiving via receiving means the response signal from the soil;
providing configuration parameters of the transmitting means and receiving means;
determining a soil structure of the soil by a subsurface model (i.e. a mathematical model describing the signal response of the subsurface model) based on the soil data (representing physical soil characteristics) and the configuration parameters;
determining a seeding, planting and/or fertilizing depth in the determined soil structure; and
providing at least one seed, plant and/or fertilizer in the determined seeding, planting and/or fertilizing depth in the soil.

In a further aspect of the present disclosure, a device for seeding, planting and/or fertilizing is presented, comprising:
a soil data providing unit configured to provide soil data by transmitting via a transmitting unit a transmitting at least one transmitting signal into the at least a part of a soil and by receiving via receiving units the response signal from the soil;
a configuration parameter providing unit configured to provide configuration parameters of the transmitting means and receiving means;
a soil structure determination unit configured to determine the soil structure of the soil by a subsurface model based on the soil data and the configuration parameters;
a seeding, planting and/or fertilizing depth determination unit configured to determine seeding, planting and/or fertilizing depth in the determined soil structure; and
a seed, plant and/or fertilizer providing unit configured to provide at least one seed, plant and/or fertilizer in the determined seeding, planting and/or fertilizing depth in the soil.

In a further aspect, a system for seeding, planting and/or fertilizing is presented, comprising
a vehicle configured to move over a soil area; and
a device for seeding, planting and/or fertilizing as described attached to the vehicle.

In a further aspect, a use of control data obtained by the method disclosed herein for operating a device for seeding, planting and/or fertilizing is presented.

In a further aspect a computer program element, in particular a computer program product or a computer readable medium, with instructions, which when executed on computing device(s) is configured to carry out the steps of any of the method disclosed herein in a system disclosed herein is presented.

This and embodiments described herein relate to the method, the system, the treatment device, the computer program element lined out above and vice versa. Advantageously, the benefits provided by any of the embodiments and examples equally apply to all other embodiments and examples and vice versa.

As used herein “determining”also includes “estimating, calculating, initiating or causing to determine”, “generating” also includes “initiating or causing to generate”, and “providing” also includes “initiating or causing to determine, generate, select, send or receive”.

The method, device, system, computer program element, disclosed herein provide an efficient, sustainable and robust way for increasing the harvest/crop yield of an agricultural field. In particular, the efficient, sustainable, and robust way for increasing can be provided by determining a seeding, planting, and/or fertilizing depth by using data from a subsurface model. Therefore, seeds, plants and/or fertilizers can be provided at depth allowing an improved growth and therefore a higher amount of crop yield. For crops like corn, soy, and cotton higher use-efficiency of the fertilizer may be obtained when the fertilizer is placed in the soil with a certain distance in the horizontal and vertical direction between seedling and fertilizer grain. Since nutrients are taken up through the roots, an optimal vertical distance between seed and fertilizer provides the required nutrition at the right moment for strong emergence and growing of the crops. Thus, an optimal placement of seed and fertilizer in the soil enables crop growth in best conditions. Compared to fertilizer spread at the surface where losses due to leaching, immobilization, and other environmental circumstances occur, up to 95% better use-efficiency can be obtained such that less fertilizer is required for best yields. The varying depth ensures seed and fertilizer placement in the best growing conditions. This means that the seeds germinate simultaneously at field level. With homogeneous germination follows homogeneous emergence as well as growth and senescence, which in turn means easier job step of harvesting.

The present disclosure provides a possibility to show the soil depth and indicate differences within the soil horizons with depth and to use this information when determining a seeding, planting and/or fertilizing depth in the determined soil structure. Changes in soil depth and layering such as depth to soil to bedrock and/or changes in soil types from top to bottom, has much impact on crop growth. Since shallow soil depth may limit root growth that influences/limits yield, it may be possible to apply/provide an on-the-go adjustment of seeding density (population) and/or fertilizer amount and placement. A further aspect is that soil depth is an important information for crop and yield modeling tools. Nowadays, the crop modeling tools/algorithms often apply a fixed soil depth (e.g. 1.8 m) over the whole field. Knowing the real soil depth drastically improves crop modeling and yield prediction tools toward more realistic outcomes. A further aspect is that one can determine water storing capacity and water draining ability of the soil horizons when knowing the horizon depths and layering of the soil. With respect to water bearing/water shortage of shallow soil, this enables adjustment of seeding population and planting depths. In addition, knowing the soil water capacity changes over the soil profile (in the different horizons with depth), this is very useful information for water balance models such that this enables irrigation management.

It is an object of the present invention to provide an efficient, sustainable and robust way for increasing the harvest/crop yield of an agricultural field and/or sustainably use fertilizer inputs. These and other objects, which become apparent upon reading the following description, are solved by the subject matters of the independent claims. The dependent claims refer to preferred embodiments of the invention.

The term soil as used herein is to be understood broadly in the present case and presents any area or volume, i.e. surface and/or subsurface, of an agricultural field to be treated by e.g. seeding, planting and/or fertilizing. The agricultural field may be any plant or crop cultivation area, such as a farming field, a greenhouse, or the like. A plant may be a crop, a weed, a volunteer plant, a crop from a previous growing season, a beneficial plant or any other plant present on the agricultural field. The agricultural field may be identified through its geographical location or geo-referenced location data. A reference coordinate, a size and/or a shape may be used to further specify the agricultural field.

The term seeding as used herein is to be understood broadly in the present case and presents any action to put, place or bring in seeds in a soil volume of an agricultural field. Seed, e.g. seed grain or seed fruit, is any biological plant material that refers to dry, dormant, generative reproductive organs such as seeds, fruits, pseudo-fruits, infructescences or parts thereof. Seeds contain the complete germ system of the plants, created by fertilization, and contains germination and growth potential.

The term planting as used herein is to be understood broadly in the present case and presents any action to put, place or bring in grown plants and/or seedlings in a soil volume/area of an agricultural field. Seedlings are young sporophyte developing out of a plant embryo from a seed. In an example, the plant is corn, soybean, cotton, sunflower, oil seed rape, wheat and/or barley.

The term fertilizing as used herein is to be understood broadly in the present case and presents any action to put, place or bring in fertilizers in a soil volume/area of an agricultural field, wherein this term also encompasses applying fertilizer additives, nitrification inhibitors, denitrification inhibitors and/or urease inhibitors. A fertilizer is any material of natural or synthetic origin that is applied to soil or to plant tissues to supply plant nutrients. In an example, the fertilizer is a starter fertilizer or a granular fertilizer.

The term geophysical method as used herein is to be understood broadly in the present case and presents any method using geophysical measuring methods. Normally geophysics measurement methods investigate/research parts of the Earth that are not or easily accessible for in situ measurements. The details of the measurement and evaluation methods used vary greatly depending on the measured variable under investigation (gravitational acceleration, electric or magnetic field strength, etc.), the observed frequency range, and the fundamental field characteristics that occur (potential field, diffusion field or wave field). For instance, geophysical methods may be potential methods like geoelectrics, geomagnetics, geothermal, and gravimetry. Diffusion methods like electromagnetic induction, magnetotellurics, geoelectromagnetics, and Very Low Frequency, acoustic wave propagation methods like seismology, seismics, high frequency electromagnetic wave propagation methods like georadar but are not limited thereto.

The term soil data as used herein is to be understood broadly in the present case and presents any data indicating and/or describing the soil. Soil data are generally provided by at least one of the above mentioned geophysical methods, wherein, depending on the chosen geophysical methods, the soil data may indicate and/or describe the physical, chemical and/or biological properties/characteristics of the soil. Exemplary, the soil data may be a dataset, when using geoelectric method, of a plurality of voltage meterings and/or resistance values being determined by using the Ohm's formula and/or a sophisticated derivation thereof incorporating configuration parameters and physical soil characteristics but is not limited thereto. The soil data may be 2-dimensional or 3-dimensional data/datasets. High-resolution 3-dimensional data of the layered subsurface may enable and improve further use-cases. Data of the complete soil profile enable precise modeling of crop growth and prediction on expected yield, improve hydrologic modeling of lateral and vertical water flow and preferential flow paths for better irrigation management, modeling of weed pressure to better steer spatially variable applications of herbicides, and enables better prescription maps for seeding and nutrition for the next season.

The term transmitting means as used herein is to be understood broadly in the present case and presents any device, apparatus, or means that transmits a signal, in particular physical signal, into the soil. Exemplary, a transmitting means may be, when using a geoelectric method, at least one pair of electrodes for providing electrical power to the soil. The transmitting means transmit the signal into at least a part of the soil, wherein the transmitted signal is preferably transmitted into a depth of: at least 1 mm, at least 2 mm, at least 5 mm, at least 1 cm, at least 2 cm, at least 5 cm, at least 25 cm, at least 75 cm, 100 cm or 150 cm.

The term receiving means as used herein is to be understood broadly in the present case and presents any device, apparatus, or means that receives Earth response signal, e.g., from the soil. Exemplary, a receiving means may be, when using a geoelectric method, at least one pair of electrodes for measuring the voltage due to the applied current in the soil.

The term configuration parameter as used herein is to be understood broadly in the present case and presents any parameter of the configuration of the transmitting means, the receiving means and/or the measuring set up. For instance, when using a geoelectric method, configuration parameters may be a distance between the receivers and transmitters, a receiver and transmitter arrangement like Wenner-arrangement, Schlumberger arrangement, dipol-dipol arrangement, pol-dipol arrangement, a receiver and transmitter orientation, a receiver and transmitter frequency, a number of used receivers and transmitters, the electrical conductivity ranges of inversion layers, thickness ranges of inversion layers, the used kind of current, i.e. AC or DC, and error values of the transmitting means and/or receiving means, but is not limited thereto.

The term subsurface model as used herein is to be understood broadly in the present case and presents any model modeling, determining, estimating, predicting and/or providing a structure of the subsurface of the soil. The subsurface model may receive the provided soil data of at least one of the above mentioned geophysical methods and the provided configuration parameter, uses these provided soil data and parameter in order to model, determine, estimate, predict a soil structure, and provides the determined soil structure. The results of the subsurface model may be 2-dimensional or 3-dimensional. For instance, when using a geoelectric method, the subsurface model receives the data of voltage meterings as soil data and the configuration parameters of the measuring setup of the geoelectric method, uses these data and parameters for determining the electrical conductivity of a soil layer and the electrical interaction among layers, and therefore identifying the soil structure of the subsurface of the soil. Further, the subsurface model provides this soil structure. To model Earth response, i.e., the response of each individual subsurface layer, due to the physical excitation of the transmitter signal, a mathematical description, e.g., differential equations, are used in a so called forward model. To adjust the forward result to the measured signal, a global optimization inversion scheme and/or a deterministic inversion scheme can be used, but is not limited thereto.

The term soil structure as used herein is to be understood broadly in the present case and presents the structure of the soil being determined/provided by the subsurface model, in particular the structure of the subsurface of the soil. For instance, when using a geoelectrical method, the soil structure can be identified by the electrical conductivity having the SI-unit “S/m” (Siemens per meter), often expressed in “mS/m” (milli Siemens per meter), and can be provided as a dataset. The soil structure may include volume information, wherein term volume information as used herein is to be understood broadly in the present case and presents any information about the lateral and vertical extend of the geophysical signal propagation that can be sensed, for example, with the arrangement of the used electrodes. Further, the soil structure may comprise plane information. The term plane information as used herein is to be understood broadly in the present case and presents any information about the soil horizons in the soil structure. Furthermore, the soil structure may comprise vertical information. The term vertical information as used herein is to be understood broadly in the present case and presents any information about the thickness of the vertical layers/horizons of the soil structure.

The term seeding, planting and/or fertilizing depth as used herein is to be understood broadly in the present case and presents a depth in which the seeds, plans and or fertilizers are placed during seating, planting and/or fertilizing action. In this context, the depth may be the specific point or a range but is not limited thereto. The SI-unit of the depth is m. The seeding, planting and/or fertilizing depth may be a specific point or a range, e.g., in which the plant has the best growth conditions. Seeding depths typically range around upper 5 cm of the soil, fertilizer placements typically around 10 cm depth.

The term region as used herein is to be understood broadly in the present case presents a specific point or a range within the soil.

The term predefined growth parameters and/or dispersion parameters for specific seeds, plants and/or fertilizers is used herein is to be understood broadly in the present case. The term predefined, i.e. preset, growth parameter presents predefined parameters indicating an optimal/ideal soil, i.e. specific appearance of water content and nutritive value, leading to an optimal growth, i.e. fastest, voluminous and/or productive/fruitful growth, of the seeded or planted plant species. Beside parameters indicating the optimal soil as mentioned above, the predefined growth parameters may also include the spatial arrangement of the seeded or planted plant species on an agricultural field, i.e. arrangement in crop rows, shifted arrangement, i.e. arrangement not in crop rows, and/or distance between two seeded or planted seeds or plants. The term predefined growth parameters may be specific values or a range. The term predefined dispersion parameters presents parameters indicating an optimal/ideal arrangement of fertilizer in the soil leading to a best possible, i.e. widespread, dispersion of the fertilizer in the soil. The ideal arrangement includes the spatial arrangement from the in the soil inserted fertilizers, i.e. liquid fertilizer or solid fertilizer, with respect to each other, i.e. horizontal and/or vertical distance between the inserted fertilizers. Further, the ideal arrangement may include the spatial arrangement from the in the soil inserted fertilizers with respect to the seeds and the plants, i.e. horizontal and/or vertical distance between the inserted fertilizers and the inserted seeds or plants. Therefore, an arrangement of seeds or plants may be an arrangement, at which the seeds or plants are arranged above and shifted with respect to the fertilizer. The term distance is to be understood broadly in the present case and represents a length of the shortest connection between two objects or points. The distance includes but is not limited to the Euclidean distance or geodesic distance.

The term control data as used herein is to be understood broadly in the present case and relates to any data configured to operate and control a device and/or a system. The control data are provided by a control unit and may be configured to control one or more technical means of the device and/or the system, e.g. the drive control and to control frequency, depth, and location of seeds, plants and/or fertilizers but is not limited thereto.

The term device for seeding, planting and/or fertilizing is to be understood broadly in the present case and comprises any device configured to provide a seed, plant and/or fertilizer into a soil of an agricultural field. The device may be any seeding, planting and/or fertilizing mechanism like a roll, a double disc coulter, an elastic arm, a robotic arm, in particular a single-or multi-articulated robot arm, or a stiff arm at which at least one outlet of the seed, plant and/or fertilizer is arranged, but is not limited thereto. The device may be arranged on a vehicle.

The term vehicle is to be understood broadly in the present case and comprises any device being configured to provide/spread seeds, plants and/or fertilizers onto the soil of an agricultural field. The vehicle may be configured to traverse the agricultural field. The vehicle may be a ground or an air vehicle, e.g. a rail vehicle, a robot, an aircraft, an unmanned aerial vehicle (UAV), a drone, or the like. The vehicle can be an autonomous or a non-autonomous vehicle.

In an embodiment of the method for seeding, planting and/or fertilizing, the providing of the soil data by transmitting and the receiving is based on a geophysical method. The use of a geophysical method provided accurate and precise, i.e. having small error values, soil data, indicating the current/present composition and/or texture of the soil both horizontally and vertically. Therefore, precise and accurate soil data can be provided.

In a further embodiment of the method for seeding, planting and/or fertilizing, the soil structure comprises volume information, plane information and/or vertical information. By comprising volume information, plane information and/or vertical information in the soil structure, distributions of soil regions having different compositions of nutrients and/or water content can be identified in 2-dimensional and 3-dimensional data.

In a further embodiment of the method for seeding, planting and/or fertilizing, the subsurface model is a forward model, a global optimization model and/or an inversion scheme.

In a further embodiment of the method for seeding, planting and/or fertilizing, wherein the determination of the seeding, planting and/or fertilizing depth includes the steps of receiving provided predefined growth parameters and/or dispersion parameters for specific seeds, plants and/or fertilizer to be seeded, planted and/or fertilized; and identifying regions in the determined soil structure which matches to the provided predefined growth parameters and/or dispersion parameters for specific seeds, plants and/or fertilizer to be seeded, planted and/or fertilized. By matching the determined soil structure with provided predefined growth parameters and/or dispersion parameters for specific seeds, plants and/or fertilizer regions or points can be indicated in which an optimal grow of the seeds or plants is provided and therefore the highest amount of crop yield can be reached.

In a further embodiment of the method for seeding, planting and/or fertilizing, the determination of the seeding, planting and/or fertilizing depth is performed for each piece of the seed, plants and/or fertilizers separately. The separate determination for each piece of the seed, plants and/or fertilizers enables that seeds, plants and/or fertilizers are arranged in such a manner that an optimal grow of the seeds or plants is provided and therefore the highest amount of crop yield can be reached.

In a further embodiment of the method for seeding, planting and/or fertilizing, the method further comprises the step of providing control data based on the determined seeding, planting and/or fertilizing depth for controlling an agricultural device. The providing of control data enables an indirect or direct controlling of a device sowing, planting and/or fertilizing device.

In a further embodiment of the method for seeding, planting and/or fertilizing, the method is carried out in real time during a run over the soil area. The carry out of the method in real time enables that calculations, determinations and/or analysis does not have to be made prior to the seeding, planting and/or fertilizing. Therefore, the treatment of an agricultural field can be made more effective and cost saving. The term real time is to be understood broadly and means in particular that during a run over with an agricultural device, the soil data are gathered and more or less directly thereafter, the soil data are processed/used to determine the seeding, planting and/or fertilizing depth, which is in turn used to provide control data for seeding, planting and/or fertilizing.

In a further embodiment of the method for seeding, planting and/or fertilizing, in a first run over, the soil data are provided and in a second run over, at least one seed, plant and/or fertilizer is provided in the soil in the determined seeding, planting and/or fertilizing depth.

In other words, the soil data may be gathered in advance, whereby correlating spatial data are added to the soil data and/or the soil data may comprise such spatial data, e.g. Global Navigation Satellite System (GNSS), Global Positioning GPS data. Assuming that multi-receiver data were measured from an external service, this data may be used to model the high-resolution three-dimensional data of the layered subsurface in the cloud and use the result for a precise prescription map for the seeding and nutrition task, for crop growth and yield modeling, for hydrologic modeling and thus water management as well as for spatially explicit weed control.

In a further embodiment of the method for seeding, planting and/or fertilizing, the method is further comprising: storing the determined seeding, planting and/or fertilizing depth data and/or the provided soil data in memory means. In an example, the soil data may be provided as comma-separated values (CSV) files.

In a further embodiment of the device for seeding, planting and/or fertilizing, the device further comprises one of the following features: a control unit configured for providing control data based on the determined seeding, planting and/or fertilizing depth for controlling an agricultural device, and a receiving unit configured for receiving provided predefined growth parameters and/or dispersion parameters for specific seeds, plants and/or fertilizer to be seeded, planted and/or fertilized and an identification unit for identifying regions in the determined soil structure which matches to the provided predefined growth parameters and/or dispersion parameters for specific seeds, plants and/or fertilizer to be seeded, planted and/or fertilized.

### BRIEF DESCRIPTION OF THE DRAWINGS

In the following, the present disclosure is further described with reference to the enclosed figures:

FIG. 1 illustrates a flow diagram of a computer-implemented method for seeding, planting and/or fertilizing;

FIG. 2 illustrates a flow diagram of a further computer-implemented method for seeding, planting and/or fertilizing of FIG. 1;

FIG. 3 illustrates a flow diagram of a further computer-implemented method for seeding, planting and/or fertilizing of FIG. 2;

FIG. 4 is a schematic illustration of a device for seeding, planting and/or fertilizing;

FIG. 5 is a schematic illustration of a device for seeding, planting and/or fertilizing of FIG. 4;

FIG. 6 illustrates a schematic illustration of a system for seeding, planting and/or fertilizing;

FIG. 7 illustrates exemplarily the different possibilities to receive and process field data; and

FIG. 8 illustrates a determined soil structure with shown determined seeding and fertilizing depth.

### DETAILED DESCRIPTION OF EMBODIMENT

The following embodiments are mere examples for implementing the method, the system or application device disclosed herein and shall not be considered limiting.

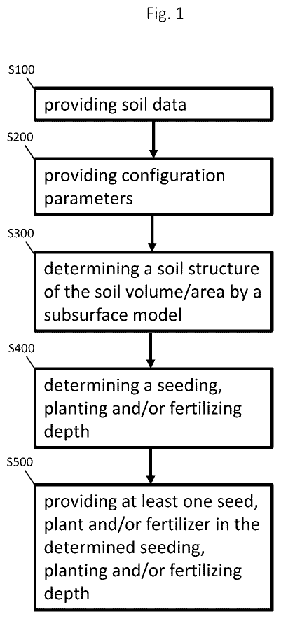

FIG. 1 illustrates a flow diagram of a computer-implemented method for seeding, planting and/or fertilizing. In the following, an exemplary order of the steps according to the present disclosure is explained. However, the provided order is not mandatory, i.e. all or several steps may be performed in a different order or simultaneously.

The method steps shown in FIG. 1 may be executed by the systems.

In a first step S100, soil data are provided by transmitting means transmitting at least one transmitting signal into at least a part of a soil and receiving maned receiving at least one response signal from the soil. The transmitting means and the receiving means are means from a geoelectric method. The transmitting means is at least one pair of electrodes being inserted into the soil and providing electrical power to the soil. The inserted electrodes may provide AC or DC power providing an electrical field in the soil. The term receiving means is at least one sensor measuring the voltage respectively the electrical field in the soil.

In a second step S200, configuration parameters of the transmitting means and receiving means are provided. The configuration parameters are the configuration of the transmitting means, the receiving means and/or the measuring set up. The configuration parameters are a distance between the receivers and transmitters, error values of the transmitting means and/or receiving means, a receiver and transmitter arrangement like Wenner-arrangement, Schlumberger arrangement, dipol-dipol arrangement, pol-dipol arrangement, a receiver and transmitter orientation, a receiver and transmitter frequency, a number of used receivers and transmitters, the number electrical conductivity ranges of inversion layers, thickness ranges of inversion layers, and/or the used kind of current, i.e. AC or DC.

In a third step S300, a soil structure of the soil area/volume is determined by a subsurface model based on the soil data and the configuration parameters. The subsurface model receives the provided soil data and the parameters as an input, uses these provided soil data and parameters in order to model, determine, estimate, predict a soil structure, and provides the determined soil structure as an output. The determined soil structure is 2-dimensional or 3-dimensional and provides the soil structure by the electrical conductivity having the SI-unit “ms/m”. The subsurface model is global optimization model. The soil structure being determined by the subsurface model presents structure of the subsurface of the soil.

In a fourth step S400, a seeding, planting and/or fertilizing depth in the determined soil structure is determined. The seeding, planting and/or fertilizing depth is the depth in which the seeds, plans and or fertilizers are placed during seating, planting and/or fertilizing action. In this context, the depth is a specific range in which the plant has the fruit-fullest growth.

In a fifth step S500, at least one seed, plant and/or fertilizer in the determined seeding, planting and/or fertilizing depth in the soil volume is provided.

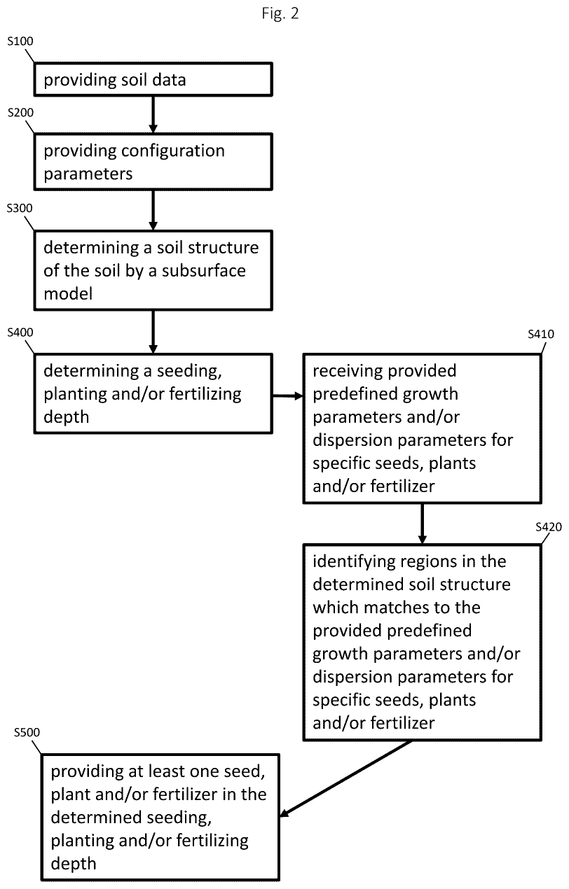

FIG. 2 illustrates a flow diagram of a further computer-implemented method for seeding, planting and/or fertilizing of FIG. 1;

The step S400 of determining a seeding, planting and/or fertilizing depth in the determined soil structure of FIG. 1 includes, in the further embodiment of the computer implemented method as depicted in FIG. 2, the sub-steps S410 receiving provided predefined growth parameters and/or dispersion parameters for specific seeds, plants and/or fertilizer to be seeded, planted and/or fertilized and S420 identifying regions in the determined soil structure which matches to the provided predefined growth parameters and/or dispersion parameters for specific seeds, plants and/or fertilizer to be seeded, planted and/or fertilized. The provided predefined growth parameters include specific parameters indicating an optimal/ideal soil, i.e. specific appearance of water content and nutritive value, leading to an optimal growth, i.e. fastest, voluminous and/or productive/fruitful growth, of the seeded or planted plant species. When matching the determined soil structure with the predefined growth parameters regions were identified in which the plants optimally growth. Based on the matching seeds, plants and/or fertilizers can be arranged only in the regions fully fulfilling the matching, i.e. having a correlation with a value of 1, or seeds, plants and/or fertilizers can be also arranged in regions having the highest possible matching, i.e. correlation with a value of 0.5 to 0.99. For instance, when seeds are seeded in a crop row in a continuously distance, the seeds are always arranged in the depth at which the regions having the highest possible matching are.

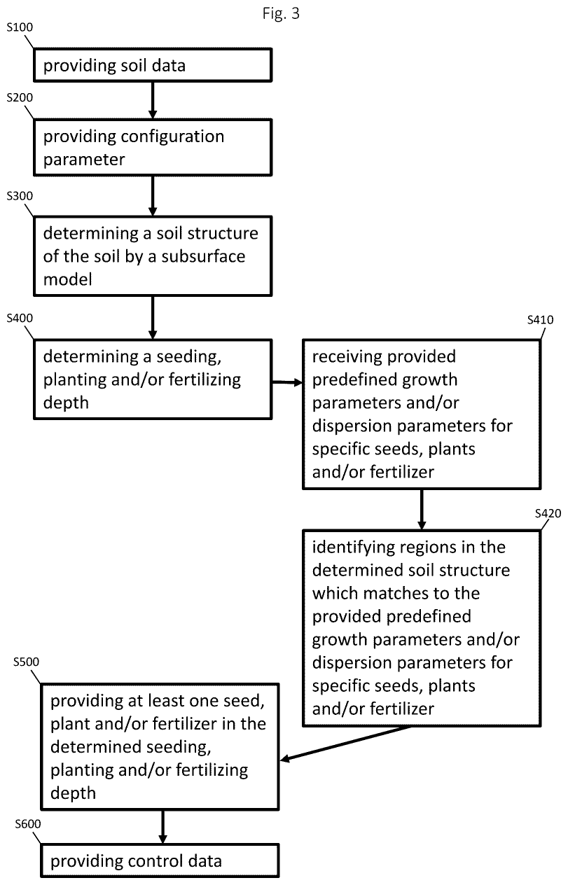

FIG. 3 illustrates a flow diagram of a further computer-implemented method for seeding, planting and/or fertilizing of FIG. 2.

Beside the steps one to five S100-S500 of FIG. 2, the further embodiment of the computer implemented method as depicted in FIG. 3 comprises a further step S600 of providing control data based on the determined seeding, planting and/or fertilizing depth for controlling an agricultural device. The control data are provided by a control unit and are configured to control one or more technical means of the device and/or the system, e.g. the drive control and to control frequency, depth, and location of seeds, plants and/or fertilizers.

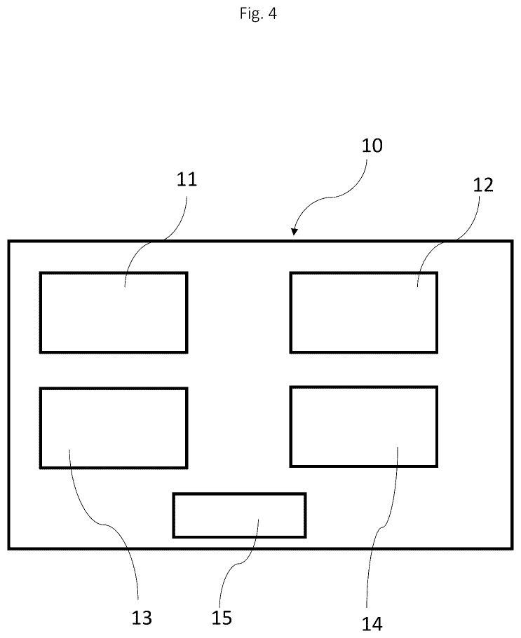

FIG. 4 is a schematic illustration of a device for seeding, planting and/or fertilizing.

The device 10 for seeding, planting and/or fertilizing comprises a first providing unit 11 for providing soil data by transmitting via a transmitting unit at least one transmitting signal into at least a part of a soil area/volume and by receiving via a receiving unit at least one response signal from the soil. The first providing unit 11 provides the soil data to the device 10 for further proceeding. The device 10 further comprises a second providing unit 12 for providing configuration parameter of the transmitting means and receiving means. The second providing unit 12 provides the configuration parameter to the system for further proceeding. Furthermore, the device comprises a first determination unit 13 and a second determination unit 14. The first determination unit 13 receives the soil data and the configuration parameter, uses these data and parameter in order to determine a soil structure of the soil by a subsurface model, and provide the determined soil structure to the device 10 for further proceeding. The second determination unit 14 receives the soil structure, uses the soil structure in order to determine a seeding, planting and/or fertilizing depth, and provides the seeding, planting and/or fertilizing depth to the device 10 for further proceeding. Additionally, the device 10 comprises a third providing unit 15, which receives the determined seeding, planting and/or fertilizing depth and uses this depth in order to provide at least one seed, plant and/or fertilizer in the determined seeding, planting and/or fertilizing depth in the soil.

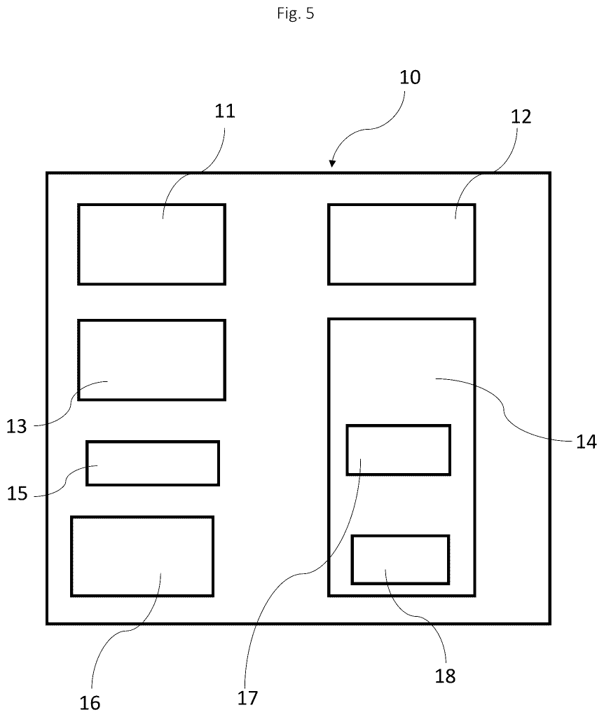

FIG. 5 is a schematic illustration of a device for seeding, planting and/or fertilizing of FIG. 4.

Beside the features as depicted in FIG. 4, the device 10 for seeding, planting and/or fertilizing further comprises a control unit 16. Further, beside the features as depicted in FIG. 4, the second determination unit 14 comprises a receiving unit 17 and an identification unit 18. The control unit 16 receives the determined seeding, planting and/or fertilizing depth, uses the seeding, planting and/or fertilizing depth in order to provide control data for controlling an agricultural device, and provides the provided control data to an agricultural device. The receiving unit 17 receives predefined growth parameters and/or dispersion parameters for specific seeds, plants and/or fertilizer to be seeded, planted and/or fertilized and provides these parameters to the second determination unit 14. The identification unit 18 receives the provided predefined growth parameters and/or dispersion parameters and the determined soil structure, matches the soil structure and the parameters in order to identify regions in the determined soil structure, and provide the identified regions to the determination unit 14.

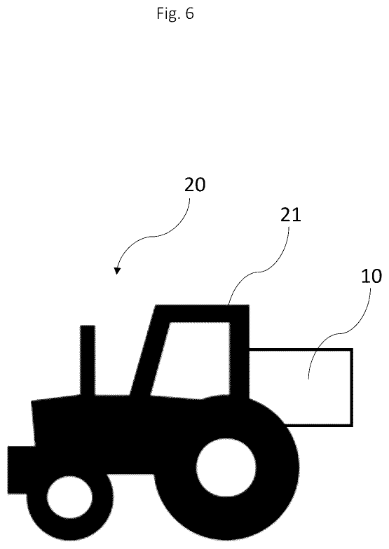

FIG. 6 illustrates a schematic illustration of a system for seeding, planting and/or fertilizing.

The system 20 comprises a vehicle 21, in particular a tractor, and a device 10 for seeding, planting and/or fertilizing, wherein the device 10 is arranged directly at the vehicle 21.

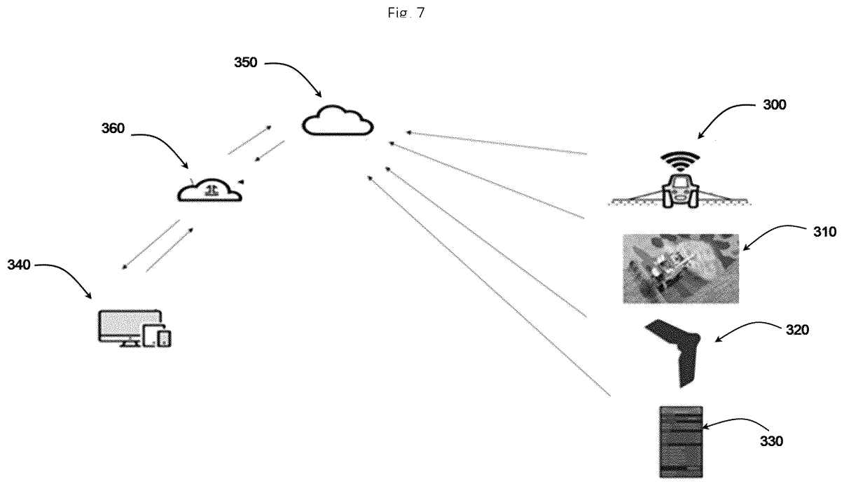

FIG. 7 illustrates exemplarily the different possibilities to receive and process field data. For example, field data can be obtained by all kinds of agricultural equipment 300 (e.g. a tractor 300) as so-called as-applied maps by recording the application rate at the time of application. It is also possible that such agricultural equipment comprises sensors (e.g. optical sensors, cameras, infrared sensors, soil sensors, etc.) to provide, for example, a weed distribution map. It is also possible that during harvesting the yield (e.g. in the form of biomass) is recorded by a harvesting vehicle 310. Furthermore, corresponding maps/data can be provided by land-based and/or airborne drones 320 by taking images of the field or a part of it. Finally, it is also possible that a geo-referenced visual assessment 330 is performed and that this field data is also processed. Field data collected in this way can then be merged in a computing device 340, where the data can be transmitted and computed, for example, via any wireless link, cloud applications 350 and/or working platforms 360, wherein the field data may also be processed in whole or in part in the cloud application 350 and/or in the working platform 360 (e.g., by cloud computing).

Aspects of the present disclosure relates to computer program elements configured to carry out steps of the methods described above. The computer program element might therefore be stored on a computing unit of a computing device, which might also be part of an embodiment. This computing unit may be configured to perform or induce performing of the steps of the method described above. Moreover, it may be configured to operate the components of the above described system. The computing unit can be configured to operate automatically and/or to execute the orders of a user. The computing unit may include a data processor. A computer program may be loaded into a working memory of a data processor. The data processor may thus be equipped to carry out the method according to one of the preceding embodiments. This exemplary embodiment of the present disclosure covers both, a computer program that right from the beginning uses the present disclosure and computer program that by means of an update turns an existing program into a program that uses the present disclosure. Moreover, the computer program element might be able to provide all necessary steps to fulfill the procedure of an exemplary embodiment of the method as described above. According to a further exemplary embodiment of the present disclosure, a computer readable medium, such as a CD-ROM, USB stick, a downloadable executable or the like, is presented wherein the computer readable medium has a computer program element stored on it which computer program element is described by the preceding section. A computer program may be stored and/or distributed on a suitable medium, such as an optical storage medium or a solid state medium supplied together with or as part of other hardware, but may also be distributed in other forms, such as via the internet or other wired or wireless telecommunication systems. However, the computer program may also be presented over a network like the World Wide Web and can be downloaded into the working memory of a data processor from such a network. According to a further exemplary embodiment of the present disclosure, a medium for making a computer program element available for downloading is provided, which computer program element is arranged to perform a method according to one of the previously described embodiments of the present disclosure.

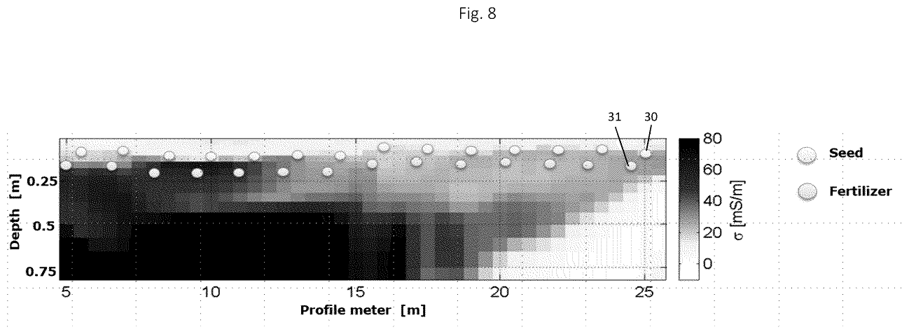

FIG. 8 illustrates a determined soil structure with shown determined seeding and fertilizing depth. The depth (y-axis of FIG. 8) extends from the surface of the soil, i.e. 0 m, to a depth of 0.75 m. The profile meter (x-axis if FIG. 8) extends from 3 m to 26 m and indicates an area of the soil being investigated. The greyscale indicates the electrical conductivity of the soil structure in the range between 0 to 80 mS/m. Further, the determined seeding and fertilizing depth of a seed 30, e.g. wheat, and a fertilizer, e.g. nitrogen fertilizer 31, in the determined soil structure. The fertilizer 31 is arranged at the fertilizer depth being deeper than the seeds 30 being arranged at the seeding depth. Further, the seeds 30 are shifted with respect to the fertilizer 31.

The present disclosure has been described in conjunction with a preferred embodiment as examples as well. However, other variations can be understood and effected by those persons skilled in the art and practicing the claimed invention, from the studies of the drawings, this disclosure and the claims. Notably, in particular, the any steps presented can be performed in any order, i.e. the present invention is not limited to a specific order of these steps. Moreover, it is also not required that the different steps are performed at a certain place or at one node of a distributed system, i.e. each of the steps may be performed at a different nodes using different equipment/data processing units.

In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation.

1. A computer-implemented method for seeding, planting and/or fertilizing, comprising:
providing soil data by transmitting via transmitting means at least one transmitting signal into at least a part of a soil and by receiving via receiving means a response signal from the soil;
providing configuration parameters of the transmitting means and receiving means;
determining a soil structure of the soil by a subsurface model based on the soil data and the configuration parameters;
determining a seeding, planting and/or fertilizing depth in the determined soil structure; and
providing at least one seed, plant and/or fertilizer in the determined seeding, planting and/or fertilizing depth in the soil.

2. The method according to claim 1, wherein the providing of the soil data by transmitting and receiving is based on a geophysical method.

3. The method according to claim 1, wherein the determined soil structure comprises volume information, plane information and/or vertical information.

4. The method according to claim 1, wherein the subsurface model is a forward model, a global optimization model and/or an inversion scheme.

5. The method according to claim 1, wherein the determination of the seeding, planting and/or fertilizing depth comprises:
receiving provided predefined growth parameters and/or dispersion parameters for specific seeds, plants and/or fertilizer to be seeded, planted and/or fertilized; and
identifying regions in the determined soil structure which matches to the provided predefined growth parameters and/or dispersion parameters for specific seeds, plants and/or fertilizer to be seeded, planted and/or fertilized.

6. The method according to claim 5, wherein the determination of the seeding, planting and/or fertilizing depth is performed for each piece of the seed, plant and/or fertilizer separately.

7. The method according to claim 1, further comprising:
providing control data based on the determined seeding, planting and/or fertilizing depth for controlling an agricultural device.

8. The method according to claim 1, wherein the method is carried out in real time during a run over the soil area.

9. The method according to claim 1, wherein in a first run over, the soil data are provided and in a second run over, at least one seed, plant and/or fertilizer is provided in the soil in the determined seeding, planting and/or fertilizing depth.

10. The method according to claim 1, wherein the method is further comprising: storing the determined seeding, planting and/or fertilizing depth data and/or the provided soil data in memory means.

11. A device for seeding, planting and/or fertilizing, comprising:
a soil data providing unit configured to provide soil data by transmitting via a transmitting unit at least one transmitting signal into at least a part of a soil and by receiving via a receiving unit a response signal from the soil;
a configuration parameter providing unit configured to provide configuration parameters of the transmitting means and receiving means;
a soil structure determination unit configured to determine the soil structure of the soil by a subsurface model based on the soil data and the configuration parameters;
a seeding, planting and/or fertilizing depth determination unit configured to determine seeding, planting and/or fertilizing depth in the determined soil structure; and
a seed, plant and/or fertilizer providing unit configured to provide at least one seed, plant and/or fertilizer in the determined seeding, planting and/or fertilizing depth in the soil.

12. The device for seeding, planting and/or fertilizing according to claim 11 further comprises at least one of the following features:
a control unit configured to provide control data based on the determined seeding, planting and/or fertilizing depth for controlling an agricultural device; and/or
a receiving unit configured to receive provided predefined growth parameters and/or dispersion parameters for specific seeds, plants and/or fertilizer to be seeded, planted and/or fertilized and an identification unit for identifying regions in the determined soil structure which matches to the provided predefined growth parameters and/or dispersion parameters for specific seeds, plants and/or fertilizer to be seeded, planted and/or fertilized.

13. A system for seeding, planting and/or fertilizing, comprising:
a vehicle configured to move over a soil area; and
a device according to claim 11 attached to the vehicle.

14. A method for controlling a device for seeding, planting and/or fertilizing comprising using the control data according to claim 7, wherein the device comprises:
a soil data providing unit configured to provide soil data by transmitting via a transmitting unit at least one transmitting signal into at least a part of a soil and by receiving via a receiving unit a response signal from the soil;
a configuration parameter providing unit configured to provide configuration parameters of the transmitting means and receiving means;
a soil structure determination unit configured to determine the soil structure of the soil by a subsurface model based on the soil data and the configuration parameters;
a seeding, planting and/or fertilizing depth determination unit configured to determine seeding, planting and/or fertilizing depth in the determined soil structure; and
a seed, plant and/or fertilizer providing unit configured to provide at least one seed, plant and/or fertilizer in the determined seeding, planting and/or fertilizing depth in the soil.

15. Computer program element with instructions, which, when executed on computing devices of a computing environment, is configured to carry out the steps of the method according to claim 1 in a system.

16. The computer program according to claim 15, wherein the system comprises a vehicle configured to move over a soil area; and a device attached to the vehicle, wherein the device comprises a soil data providing unit configured to provide soil data by transmitting via a transmitting unit at least one transmitting signal into at least a part of a soil and by receiving via a receiving unit a response signal from the soil; a configuration parameter providing unit configured to provide configuration parameters of the transmitting means and receiving means; a soil structure determination unit configured to determine the soil structure of the soil by a subsurface model based on the soil data and the configuration parameters; a seeding, planting and/or fertilizing depth determination unit configured to determine seeding, planting and/or fertilizing depth in the determined soil structure; and a seed, plant and/or fertilizer providing unit configured to provide at least one seed, plant and/or fertilizer in the determined seeding, planting and/or fertilizing depth in the soil
